(19) United States
(12) Patent Application Publication
(10) Pub. No.: US 2004/0167506 A1
(43) Pub. Date: Aug. 26, 2004

(54) Medical devices employing ferromagnetic heating

(75) Inventor: John J. Chen, Plymouth, MN (US)

Correspondence Address: David M. Crompton, CROMPTON, SEAGER & TUFTE, LLC, Suite 800, 1221 Nicollet Avenue, Minneapolis, MN, 55403-2420, US

(73) Assignee: SciMed Life Systems, Inc.

(21) Appl. No.: 10/375,719

(22) Filed: Feb. 25, 2003

(51) Int. Cl. A61B 18/04

(52) U.S. Cl. 606/27

(57) ABSTRACT

Ferromagnetic heating can be employed in medical devices such as catheters. Catheters bearing ferromagnetic materials can be used to provide localized and directed heating to a treatment site such as an intravascular treatment site. In particular, a balloon catheter can be positioned proximate an intravascular treatment and can be heated by applying an alternating magnetic field.

### TECHNICAL FIELD

The invention relates generally to medical devices and more specifically to medical devices that utilize ferromagnetic heating.

### BACKGROUND

Medical devices that can deliver heat to selected portions of a patient are known, including catheters that can deliver heat. Catheters such as balloon catheters can deliver heat through a variety of mechanisms, including recirculating a heated fluid through the balloon or through other portions of the catheter, and electro-resistive heating. A need remains for improved heat delivery means and methods.

### SUMMARY

The invention provides design, material, structural and manufacturing alternatives for medical devices that can provide heat. In some embodiments, the invention provides alternatives for medical devices such as catheters that employ ferromagnetic heating. Catheters bearing ferromagnetic materials can be used to provide localized and directed heating to a treatment site such as an intravascular treatment site. A catheter can be positioned proximate an intravascular treatment and can be heated by applying an alternating magnetic field.

In particular, an example embodiment can be found in a thermal treatment catheter that has an elongate shaft with a proximal portion and a distal portion. A ferromagnetic material can be disposed within the distal portion of the catheter.

Another example embodiment can be found in a balloon catheter that includes an elongate shaft having a proximal end and a distal end, and an inflatable balloon that is arranged near the distal end of the elongate shaft. The catheter can include a ferromagnetic material.

Another example embodiment can be found in a thermal treatment method involving a thermal treatment catheter having an elongate shaft with a distal end and a ferromagnetic heat source positioned near the distal end. The thermal treatment catheter can be positioned such that the ferromagnetic heat source is proximate a treatment site, and an alternating magnetic field can be applied to activate the ferromagnetic heat source and thus apply heat to the treatment site.

### BRIEF DESCRIPTION OF FIGURES

FIG. 1 is a plan view of a catheter in accordance with an embodiment of the invention;

FIG. 2 is a cross-sectional view of the catheter of FIG. 1, taken along line 2-2;

FIG. 3 is a partially-sectioned view of a portion of the catheter of FIG. 1;

FIG. 4 is a plan view of a balloon catheter in accordance with an embodiment of the invention;

FIG. 5 is a partially-sectioned view of a single layer balloon in accordance with an embodiment of the invention;

FIG. 6 is a partially-sectioned view of a double layer balloon in accordance with an embodiment of the invention;

FIG. 7 is a partially-sectioned view of a modified double layer balloon in accordance with an embodiment of the invention;

FIG. 8 is a plan view of a balloon catheter positioned over a guidewire, proximate a lesion within a blood vessel, illustrating a use of the catheter in accordance with an embodiment of the invention;

FIG. 9 is a plan view of the balloon catheter of FIG. 8, showing the balloon in its inflated configuration; and

FIG. 10 is a plan view of the blood vessel of FIGS. 8 and 9, showing the lesion after compaction and after catheter withdrawal.

### DETAILED DESCRIPTION

Medical devices such as catheters bearing ferromagnetic materials can be used to provide localized heating to a treatment site such as an intravascular treatment site. A catheter or other medical device can be positioned proximate an intravascular treatment site and can be heated by applying an alternating magnetic field.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, any reference to “percent” or “%” are intended to be defined as weight percent, unless explicitly described to the contrary.

The following description should be read with reference to the drawings wherein like reference numerals indicate like elements throughout the several views. The detailed description and drawings illustrate example embodiments of the claimed invention.

The invention pertains to employing ferromagnetic heating to deliver therapeutic amounts of thermal energy to a desired target location on or within a patient's body. There can be a number of therapeutic or treatment purposes in providing heat to a desired target location. For example, if the target location is an intravascular lesion such as plaque buildup or an intravascular occlusion, heat can be useful in molding or shaping the lesion after it has been compressed. In some situations, heat can be useful to soften the lesion prior to balloon inflation. If sufficient heat is applied, in some circumstances tissue growth can be depressed. Tissue ablation is also possible, given appropriate time and temperature parameters.

In broad terms, ferromagnetic heating refers to an inductive form of heating in which an alternating magnetic field can cause susceptors such as ferromagnetic particles to increase in temperature. In particular, when a ferromagnetic material is placed within an alternating magnetic field, it heats due to hysteresis loss. The heat generated can be transferred to a target position on or within the patient via conduction and/or convection. An advantage of using ferromagnetic heating is that all ferromagnetic materials have a Curie temperature, above which they become paramagnetic and no longer heat. A desired heating temperature can be reached by controlling characteristics such as the type of ferromagnetic particle, the particle size and the volume fraction of the ferromagnetic material. Particular ferromagnetic materials will be described hereinafter.

Controlled heat application via ferromagnetic heating can be employed in a variety of different medical devices that are intended for a variety of different interactions and applications on and within a patient's body. For illustrative but non-limiting purposes, the invention will be described with reference to intravascular heating employing catheters such as balloon catheters. The scope of the invention is not limited to such, however. Other examples of catheters include balloon angioplasty catheters, stent delivery catheters, artheroectomy catheters, guide catheters and drug delivery catheters.

FIG. 1 illustrates a catheter in accordance with an embodiment of the present invention. In particular, FIG. 1 is a sectional side view of a catheter 10 that has a proximal end 12 and a distal end 14. A manifold 16 is positioned at the proximal end 12 and is connected to a catheter shaft 18 and includes a strain relief 20. The manifold 16 generally contains port 22 that allows for fluid-tight connections. A luer-lock fitting is an example of a fluid-tight fitting attached to the manifold port 22.

The distal end 14 of the catheter 10 can be arranged and configured depending on the intended use for the catheter 10. In some embodiments, the catheter 10 can include a soft tip (not illustrated) made of a soft material that minimizes trauma to the surrounding tissue as catheter 10 is advanced to, and ultimately engaged with, its final destination within the vasculature.

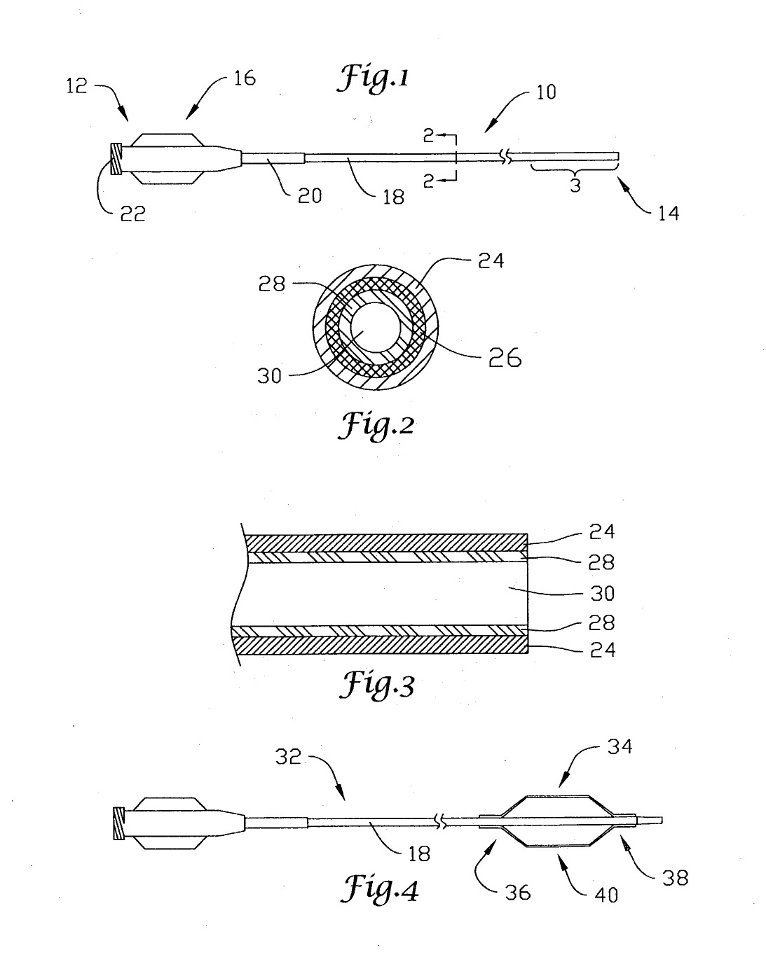

The catheter shaft 18 is best illustrated in reference to FIGS. 2 and 3. FIG. 2 is a cross-sectional view of the catheter shaft 18, taken along line 2-2 of FIG. 1. As illustrated, the catheter shaft 18 includes an outer layer or sleeve 24, an intermediate reinforcing layer 26 and an inner layer 28. The catheter shaft 18 defines a lumen 30 that is disposed within and defined by the inner layer 28. Except as described herein, construction of the multi-layer catheter shaft 18 is conventional. The inner layer 28 can be a conventional lubricious polymer layer while the outer layer 24 can be a conventional polymer layer.

Examples of possible polymeric materials that can be used in forming the outer layer 24 and the inner layer 28 include, but are not limited to, poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), poly D,L-lactide-co-caprolactone) (PLA/PCL), poly(glycolide-co-caprolactone) (PGA/PCL), polyanhydrides (PAN), poly(ortho esters), poly(phosphate ester), poly(amino acid), poly(hydroxy butyrate), polyacrylate, polyacrylamid, poly(hydroxyethyl methacrylate), polyurethane, polysiloxane, aromatic and aliphatic polyketone, polyethersulfone, polysulfone, acetal, polycarbonate, polyetherimide, polyethylene, polypropylene, polyamide, polyesters and their copolymers.

In some embodiments, the intermediate reinforcing layer 26 can extend from the proximal end 12 to the distal end 14 of the catheter 10. In some embodiments, the intermediate reinforcing layer 26 can extend from a point at or near the proximal end 12 of the catheter to a point that is proximal of the distal end 14 of the catheter 10. This is illustrated in part in FIG. 3, which shows a lumen 30 that is defined by an inner layer 28 and an outer layer 24. In some embodiments, distal flexibility is more important than column strength, and thus, the intermediate reinforcing layer 26 can, as noted above, stop proximal of a distal portion of the catheter 10.

One or both of the outer layer 24 and the inner layer 28 can include a ferromagnetic material. The ferromagnetic material can be dispersed within a polymer that forms the outer layer 24 or the inner layer 28. In some embodiments, the ferromagnetic material is dispersed within the polymer forming the outer layer 24. The ferromagnetic material can be provided within the outer layer 24 in particulate form, having an average particle size that is in the range of about 0.1 micron to about 500 microns.

In some embodiments, the ferromagnetic material can reach a temperature of at least about 45° C. when subjected to an alternating magnetic field at a frequency of less than about 10 MHz. In particular embodiments, the ferromagnetic material can react to a magnetic field that alternates at a frequency of about 275 kHz. In preferred embodiments, the magnetic field alternates at a frequency in the range of 200 kHz to 10 MHz. The ferromagnetic material can be selected to have a heating temperature that is in the range of about 100° C. to about 600° C. The device heating temperature can be controlled by adjusting particle material, particle size and particle distribution.

In particular embodiments, the outer layer 24 can be a polysulfone film that contains about 30 weight percent ferromagnetic material. In such embodiments, the outer layer 24 can be in the range of about 0.5 mils to 5 mils thick. Particular ferromagnetic materials that are useful in the practice of the invention include SrFe₁₂O₁₉, Co₂Ba₂Fe₁₂O₂₂, and Fe₃O₄. While not illustrated, the ferromagnetic material also can be included in a thin film such as the aforementioned polysulfone film that can be provided over the outer layer 24.

Depending on the intended use of the catheter 10, the ferromagnetic material can be concentrated at or near the distal end 14 of the catheter shaft 18. A concentrated distribution of the ferromagnetic material, whether in the outer layer 24 or the inner layer 28, can provide for localized pinpoint heating. In some embodiments, the ferromagnetic material can be more widely distributed within at least one of the outer layer 24 and the inner layer 28 if heating is desired over a larger area.

In particular embodiments, the catheter 10 can be a balloon catheter such as a balloon angioplasty catheter 32 as illustrated, for example, in FIG. 4. FIG. 4 is a plan view of a balloon angioplasty catheter 32 that is similar in construction to the catheter 10, but includes a balloon 34. As illustrated, the balloon 34 has a proximal waist 36, a distal waist 38 and an intermediate portion 40. The balloon 34 is seen in an expanded or inflated configuration. Construction of the balloon angioplasty catheter 32 is conventional except as described herein.

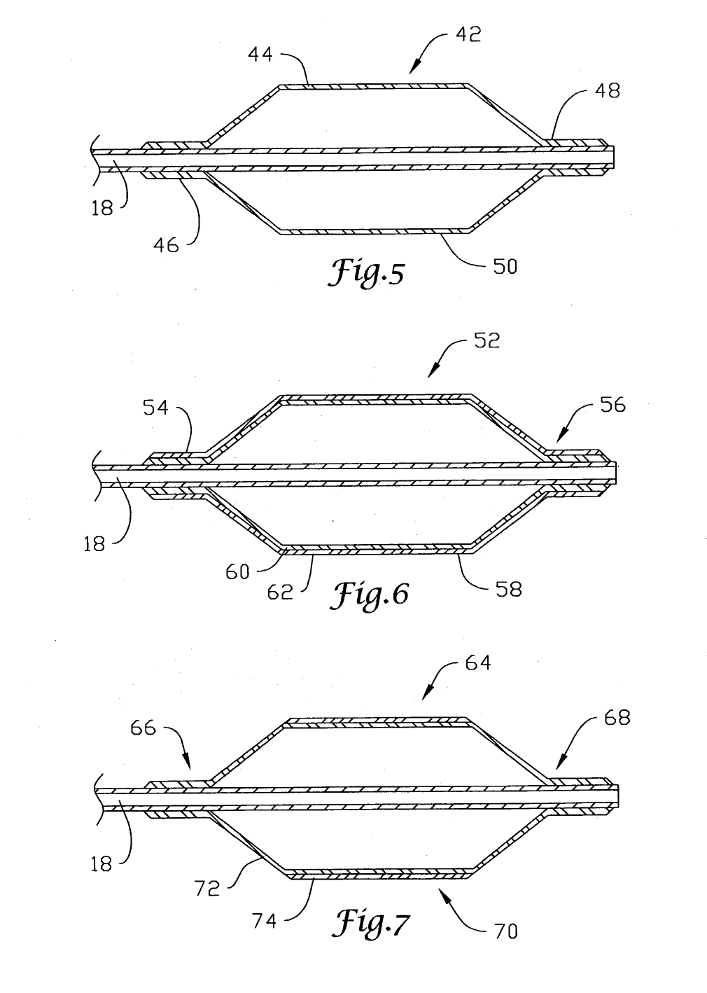

FIGS. 5, 6 and 7 illustrate particular embodiments of the balloon 34. In particular, FIG. 5 is a partially-sectioned view of a balloon 42 that is formed of a single layer 44. The balloon 42 has a proximal waist 46, a distal waist 48 and an intermediate portion 50 and can be attached to the catheter shaft 18 at the proximal and distal waists 46 and 48, respectively.

The single layer 44 can be formed of any suitable polymeric material, and can include ferromagnetic material in particulate form. In some embodiments, the ferromagnetic material can be distributed throughout the polymer forming the single layer 44. In some embodiments, the ferromagnetic material can be concentrated along the intermediate portion 50 of the balloon 42 within the single layer 44.

FIG. 6 illustrates a balloon 52 that has a proximal waist 54, a distal waist 56 and an intermediate portion 58. The balloon 52 can have an inner layer 60 and an outer layer 62 that extend from the proximal waist 54 to the distal waist 56 and can be attached to the catheter shaft 18 at the proximal waist 54 and the distal waist 56. In some embodiments, the ferromagnetic material can be dispersed within the polymer forming the inner layer 60 or the outer layer 62. In some embodiments, the ferromagnetic material can be dispersed evenly throughout one of the inner or outer layers 60 and 62, or the ferromagnetic material can be concentrated along the intermediate portion 58 within one or both of the inner and outer layers 60 and 62.

FIG. 7 shows a balloon 64 that has a proximal waist 66, a distal waist 68 and an intermediate portion 70 and can be attached to the catheter shaft 18 at the proximal waist 66 and the distal waist 68. The balloon 64 can have an inner layer 72 that extends from the proximal waist 66 to the distal waist 68 and an outer layer 74 that extends along the intermediate portion 70 of the balloon 64. In some embodiments, the ferromagnetic material can be dispersed within the polymer forming the inner layer 72 or the outer layer 74. In some embodiments, the ferromagnetic material can be dispersed evenly throughout one of the inner or outer layers 72 and 74, or the ferromagnetic material can be concentrated along the intermediate portion 58 within one or both of the inner and outer layers 72 and 74. In some embodiments, the ferromagnetic material can be distributed within the outer layer 74.

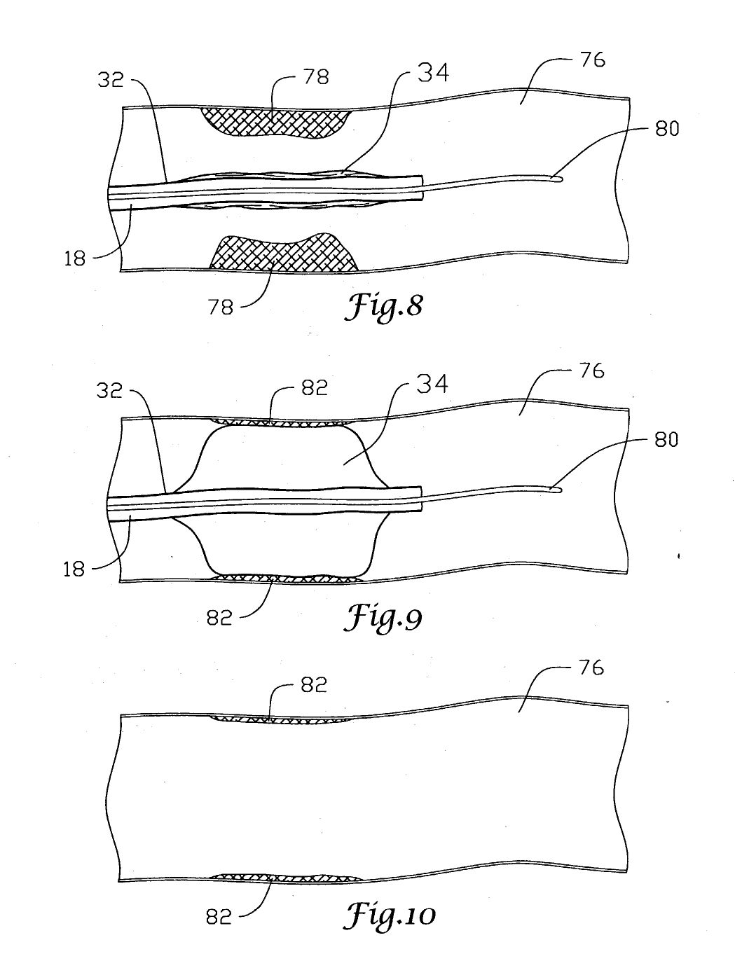

An illustrative but non-limiting use of a balloon angioplasty catheter in accordance with an embodiment of the present invention is demonstrated in FIGS. 8, 9 and 10. In FIG. 8, a balloon catheter 32 has been positioned within a blood vessel 76 proximate a lesion 78. The balloon catheter 32 is positioned over a guidewire 80 with the balloon 34 in a deflated, insertion configuration. As illustrated in FIG. 9, the balloon 34 can be inflated to compress or otherwise move or deflect the lesion 78 so that it consumes less of the volume of the blood vessel 76.

In some embodiments, an alternating magnetic field can be applied once the balloon 34 has been fully inflated and is in full contact with the lesion 78. In some embodiments, the balloon 34 can be partially inflated prior to applying an alternating magnetic field. Once the lesion 78 has been heated as a result of the hysteresis losses within the ferromagnetic material, the balloon 34 can be fully inflated. In any event, once the balloon 34 has been deflated and the balloon catheter 32 and guidewire 80 have been withdrawn, the blood vessel 76 can have increased relative volume as illustrated in FIG. 10 as a result of the lesion 78 being compressed to form a compressed lesion 82.

In some embodiments, applying heat to the lesion 78 results in softening the lesion 78 prior to partial or complete balloon inflation. In some embodiments, applying heat results in shaping or molding the lesion 78. If sufficient heat is applied, tissue within or behind the lesion 78 can be thermally deactivated or can even be ablated.

As noted, the medical devices in accordance with the present invention can be of conventional materials and construction, except as described herein. Medical devices such as the catheter 10 and the balloon angioplasty catheter 32 can be partially or completely coated with a lubricious or other type of coating. Hydrophobic coatings such as fluoropolymers provide a dry lubricity that can improve handling and device exchanges. An example of a suitable fluoropolymer is polytetrafluoroethylene (PTFE), better known as TEFLON®.

Lubricious coatings can improve steerability and improve lesion crossing capability. Examples of suitable lubricious polymers include hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers can be blended among themselves or with formulated amounts of water insoluble compounds (including some polymers) to yield coatings with suitable lubricity, bonding, and solubility. In some embodiments, a distal portion of a composite medical device can be coated with a hydrophilic polymer as discussed above, while the more proximal portions can be coated with a fluoropolymer.

The medical devices described herein, such as the catheter 10 and the balloon angioplasty catheter 32, can include, or be doped with, radiopaque material to improve visibility when using imaging techniques such as fluoroscopy techniques. Any suitable radiopaque material known in the art can be used. Some examples include precious metals, tungsten, barium subcarbonate powder, and the like, and mixtures thereof. In some embodiments, radiopaque material can be dispersed within the polymers used to form the particular medical device. In some embodiments, the radiopaque materials distinct from the ferromagnetic materials are dispersed.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

We claim:

1. A thermal treatment catheter comprising:
an elongate shaft having a proximal portion and a distal portion; and
a ferromagnetic material disposed within the distal portion of the catheter.

2. The thermal treatment catheter of claim 1, wherein the elongate shaft comprises a polymer, and the ferromagnetic material is disposed within the polymer.

3. The thermal treatment catheter of claim 1, wherein the elongate shaft comprises an inner sleeve and a coaxially disposed outer sleeve, and one of the inner sleeve and the outer sleeve includes the ferromagnetic material.

4. The thermal treatment catheter of claim 3, wherein the outer sleeve includes the ferromagnetic material.

5. The thermal treatment catheter of claim 1, further comprising an inflatable balloon positioned near the distal end of the elongate shaft.

6. The thermal treatment catheter of claim 5, wherein the ferromagnetic material is provided within the inflatable balloon.

7. The thermal treatment catheter of claim 5, wherein the inflatable balloon comprises a single layer and the ferromagnetic material is disposed within the single layer.

8. The thermal treatment catheter of claim 5, wherein the inflatable balloon comprises two layers, with a first layer forming the inflatable balloon and a second layer disposed adjacent the first layer that includes the ferromagnetic material disposed therein.

9. The thermal treatment catheter of claim 1, wherein the ferromagnetic material comprises a material that reaches a temperature of at least about 45° C. when subjected to an alternating magnetic field at a frequency less than about 10 MHz.

10. The thermal treatment catheter of claim 9, wherein the magnetic field alternates at a frequency of about 275 kHz.

11. A balloon catheter comprising:
an elongate shaft having a proximal end and a distal end; and
an inflatable balloon arranged near the distal end of the elongate shaft;
wherein the catheter comprises a ferromagnetic material.

12. The balloon catheter of claim 11, wherein the ferromagnetic material is provided within the elongate shaft.

13. The balloon catheter of claim 11, wherein the ferromagnetic material is provided within the inflatable balloon.

14. The balloon catheter of claim 13, wherein the inflatable balloon comprises a single layer and the ferromagnetic material is disposed within the single layer.

15. The balloon catheter of claim 13, wherein the inflatable balloon comprises two layers, with a first layer forming the inflatable balloon and a second layer disposed adjacent the first layer that includes the ferromagnetic material disposed therein.

16. The balloon catheter of claim 15, wherein the second layer comprises a polysulfone film that contains about 30 weight percent ferromagnetic material and that is in the range of about 0.5 to 5 mils thick.

17. The balloon catheter of claim 11, wherein the ferromagnetic material comprises a material having a heating temperature in the range of about 100° C. to about 600° C.

18. The balloon catheter of claim 11, wherein the ferromagnetic material comprises particles having an average size in the range of about 0.1 micron to about 500 microns.

19. The balloon catheter of claim 11, wherein the ferromagnetic material comprises a material selected from the group consisting of SrFe₁₂O₁₉, Co₂Ba₂Fe₁₂O₂₂, and Fe₃O₄.

20. The balloon catheter of claim 11, wherein the ferromagnetic material is distinct from any radiopaque materials added to lend radiopacity to the balloon catheter.

21. The balloon catheter of claim 11, wherein the inflatable balloon comprises a distal waist, a proximal waist, and an inflatable intermediate portion positioned therebetween, the intermediate portion bearing the ferromagnetic material.

22. The balloon catheter of claim 21, wherein the distal and proximal waists are substantially free of the ferromagnetic material.

23. A thermal treatment method comprising:
providing a thermal treatment catheter comprising an elongate shaft having a distal end and a proximal end, and a ferromagnetic heat source positioned near the distal end of the elongate shaft;
positioning the thermal treatment catheter such that the ferromagnetic heat source is proximate a treatment site; and
applying an alternating magnetic field to activate the ferromagnetic heat source, thereby applying heat to the treatment site.

24. The thermal treatment method of claim 23, wherein the treatment site comprises an intravascular lesion.

25. The thermal treatment method of claim 24, wherein applying heat to the intravascular lesion comprises applying sufficient heat to soften the lesion.

26. The thermal treatment method of claim 24, wherein applying heat to the intravascular lesion comprises applying sufficient heat to thermally deactivate tissue within or behind the lesion.

27. The thermal treatment method of claim 24, wherein applying an alternating magnetic field comprises applying a magnetic field that alternates at a frequency that is in the range of about 200 kHz to 10 MHz.
